(19) United States
(12) Patent Application Publication
(10) Pub. No.: US 2025/0099328 A1
(43) Pub. Date: Mar. 27, 2025

(54) SYSTEM FOR STORING AND DELIVERING A MEDICINAL SOLUTION INTO/OUT OF A MULTIPLE DOSE CONTAINER

(71) Applicant: Gaplast GmbH, Altenau (DE)

(72) Inventors: Stephan Kneer, Farchant (DE); Alexander Keller, Rottenbuch (DE)

(21) Appl. No.: 18/800,659

(22) Filed: Aug. 12, 2024

(30) Foreign Application Priority Data
Sep. 27, 2023 (DE) 10 2023 126 352.8

(51) Int. Cl. A61J 1/05; A61M 5/178

(52) U.S. Cl. CPC A61J 1/05; A61M 5/1782

(57) ABSTRACT

A system for storing a sterile medicinal solution in a multiple dose container and for delivering one dose at a time of the medicinal solution to a disposable syringe, where the multiple dose container is an airless container, which consists of a stiff outer container and a contracting inner bag, into which no air enters for pressure balancing when delivering medicinal solution and fastened to the multiple dose container is an adapter with a one-way valve and a female Luer container. A disposable syringe is provided with a matching, male Luer connector, with which it may be docked with the adapter to receive a dose of the medicinal solution by pulling up the disposable syringe.

### PRIORITY CLAIM

The subject application claims convention priority to German patent application No. DE 10 2023 126 352.8, filed Sep. 27, 2023.

### BACKGROUND

The invention relates to a system for storing a preferably sterile medicinal solution in a multiple dose container and for dispensing one dose at a time of the medicinal solution to a disposable syringe.

It was previously common to store a medicinal solution in a so-called perforable ampoule or a vial, which is sealed by a pierceable membrane or a rubber stopper, which has the smallest thickness in the centre. In order to remove a dose of medicinal solution, the rubber stopper or the pierceable membrane is pierced by the needle of a standard disposable syringe, whereafter by raising the syringe the dose of the medicinal solution is taken up into the syringe. There are several steps in this which are vulnerable to contamination of the medicinal solution and the syringe needle and entail the risk of faulty operation. The perforable ampoule can commonly contain up to 200 ml of the medicinal solution so that numerous doses of the medicinal solution are generally to be received into a disposable syringe and the stopper is pierced each time so that contaminants can get into the medicinal solution. Furthermore, the surface of the stopper can be contaminated. It can additionally occur that for reasons of time and cost the same needle is used for the removal from the vial and for the application to a patient. In the removal process, the perforable ampoule is to be held in an upside down position since air enters into the ampoule for pressure balance purposes at each removal. Furthermore, on each removal from the perforable ampoule there is a not inconsiderable risk of a needle prick injury.

It is the object of the present invention to provide a system in which the above disadvantages are wholly or largely avoided. It is to be ensured that the medicinal solution can be stored, preferably in a sterile manner, in a multiple dose container and that the individual doses are received into a standard disposable syringe in a few simple steps. Contamination of the medicinal solution is to be eliminated to the greatest possible extent both within the container and also during the uptake into the disposable syringe.

### SUMMARY

The invention provides that the multiple dose container is an airless container, which consists of a stiff outer container and an inner bag, into which no air enters for pressure balancing when dispensing medicinal solution. Fastened to the multiple dose container is an adapter with a one-way valve with a Luer connector, with which a Luer cone on a standard disposable syringe can dock for a simple, uncomplicated and reliable pulling up of the syringe with the medicine.

The construction is so designed that the adapter is provided with a female Luer connector whilst the disposable syringe has the matching, male Luer connector so that there is a sealed connection between the multiple dose container and the disposable syringe when pulling up the syringe.

A dead volume within the adapter and the one-way valve can additionally be provided with antimicrobial material, such as silver, so that it is ensured that no microbial contamination can get into the medicinal solution in the multiple dose container.

The reduced pressure in the multiple dose container occurring during the dispensing of medicinal solution to the disposable syringe is balanced out by air entering through an opening in the outer container into the gap between the outer container and the contracting inner bag so that no air gets into the interior of the inner bag. Completely air-free filling is thus possible.

The multiple dose container has a container neck, onto which the adapter with the Luer connector and the one-way valve is preferably snap-connected, crimped or screwed. The one-way valve preferably includes an approximately bell-shaped outer cone, which preferably consists of a thermoplastic elastomer, and seals the entire inner opening of the one-way valve with the exception of a hole, which is formed in the middle of the valve membrane, the edge of which rests, in the relaxed state of the valve membrane, on a valve seat pin, which sealingly closes the hole in the relaxed state of the membrane. Air is thus prevented from getting into the interior of the multiple dose container from the exterior.

When the disposable syringe with its Luer connector is inserted into the conically broadening outlet passage of the one-way valve and the disposable syringe is pulled up the elastic outer cone or the elastic valve membrane is lifted upwardly from the valve seat pin by the suction, whereby a dose of the medicinal solution is drawn through the hole into the syringe. If an antimicrobial substance, such as a silver ring, is advantageously arranged on the inner surface of the female Luer connector, contamination of the drawn in medicinal solution is practically eliminated.

As a further feature for ensuring an aseptic filling, it is proposed with advantage that the outlet opening in the Luer adapter is closeable by a lid, which sealingly engages on the upper surface of the outlet opening, which is connected to two lateral webs, which are pivotally connected to the outer side of the adapter, so that the lid may be swung sideways. The lid is connected furthermore to one or two flexible spring arms, which are fastened to the outer side of the adapter. The lid is movable laterally on the outlet opening against the force of the flexible spring arms in order to expose the outlet opening for the Luer connector of the disposable syringe, whereby the flexible spring arms move the lid back into its closed position again after the removal process. The lid can have an edge opening, into which the Luer connector on the disposable syringe can engage in order to move the lid laterally, which has the advantage that the lid need not be contacted by the hand of the user for this purpose. A rotatable or tiltable version of the lid is also possible.

The system in accordance with the invention ensures on the one hand that no air gets into the multiple dose container so that the medicinal solution can be aseptically filled with reduced or absolutely no preserving agent content. The one-way valve prevents air getting into the container so that microbial growth from the exterior to the interior is eliminated as well as is possible, whereby dead volumes in the one-way valve can be maintained free of contamination by silver or other antimicrobial materials. Due to the Luer connector, the pulling up of the syringe is simple, uncomplicated and reliable, whereby the risk of injury by the needle when taking up the dose is reliably avoided. When introducing the disposable syringe into the Luer adapter on the vial, an upside down position is additionally not necessary since there is no air in the interior of the vial.

### BRIEF DESCRIPTION OF THE DRAWINGS

FIG. 1A shows a side view of an embodiment of the system in accordance with the invention;

FIG. 1B shows a side view of the system of FIG. 1 rotated through 90°;

FIG. 1C shows a top view of the system of FIG. 1;

FIG. 1D is a vertical sectional view through the upper region of the system of FIG. 1;

FIG. 2A shows a fragmentary section view of the system of FIG. 1 in position 1 in the removal of a dose of the medicinal solution;

FIG. 2B shows a fragmentary section view of the system of FIG. 1 in position 2 in the removal of a dose of the medicinal solution;

FIG. 2C shows a fragmentary section view of the system of FIG. 1 in position 3 in the removal of a dose of the medicinal solution;

FIG. 2D shows a fragmentary section view of the system of FIG. 1 in position 4 in the removal of a dose of the medicinal solution;

FIG. 2E shows a fragmentary section view of the system of FIG. 1 in position 5 in the removal of a dose of the medicinal solution;

FIG. 3 is a vertical sectional view through the adapter and the one-way valve of the system of FIG. 1;

FIG. 4 shows an enlarged fragmentary front view of detail E of FIG. 3;

FIG. 5A shows a top view of another embodiment of the adapter of the present system, where the adapter includes two spring elements.

FIG. 5B shows a side view of the adapter of FIG. 5A.

### DETAILED DESCRIPTION

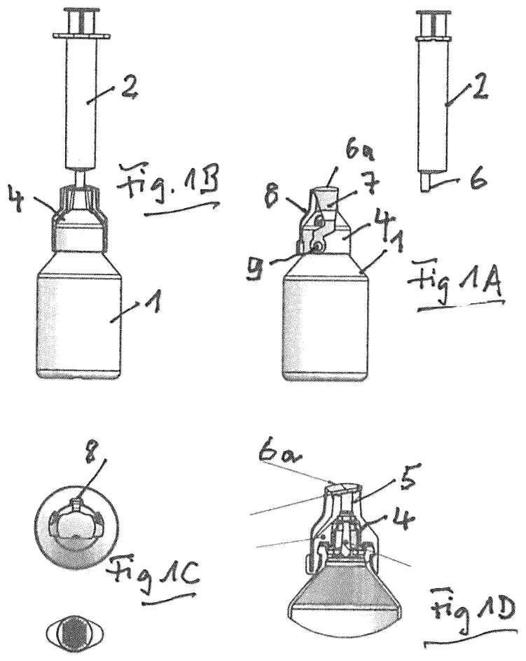

FIG. 1A is an exploded view of a multiple dose container 1, which contains a preferably sterile medicinal solution, from which one dose at a time of medicinal solution is removed with the aid of a disposable syringe 2. The multiple dose container is a so-called airless container, which consists of a stiff outer container and an inner bag, which contracts after removal of medicinal solution, for which purpose the outer container is provided with at least one hole/opening, through which air enters on each occasion for pressure balancing when medicinal solution is delivered. No air thus enters into the inner bag for pressure balancing.

The multiple dose container can have any suitable shape, for instance a cylindrical or bulbous bottle shape with a container neck.

Fastened to the container neck of the multiple dose container 1 is an adapter 4, arranged in which is a one-way valve 3, which will be described in more detail below. The outlet of the valve is constituted by a female Luer connector 5 on the adapter 4, into which a male Luer connector 6 on the disposable syringe 2 can sealingly engage in order to remove a dose of the medicinal solution from the multiple dose container 1.

The disposable syringe is conveniently introduced into the adapter 4 from above, as shown in FIG. 1B, which is possible because the medicinal solution is contained only in the inner bag.

The Luer connector 5 on the adapter 4 is tightly sealed, in the non-actuated state of the system, by a lid-like closure 6a, which is pivotally connected to the outer side of the adapter 4 by means of webs 7 on both sides and is pivotable into an open position against the force of a spring 8 in order to expose the Luer connector 5 for the disposable syringe 2. The closure 6a is pivotally connected to the adapter 4 at lateral bearing pins 9. The bent spring 8 is offset through 90° on the closure 6a and fastened to the adapter 4 and is tensioned when the closure 6a is opened in order to pivot the closure 6a back into the closed position after the removal process.

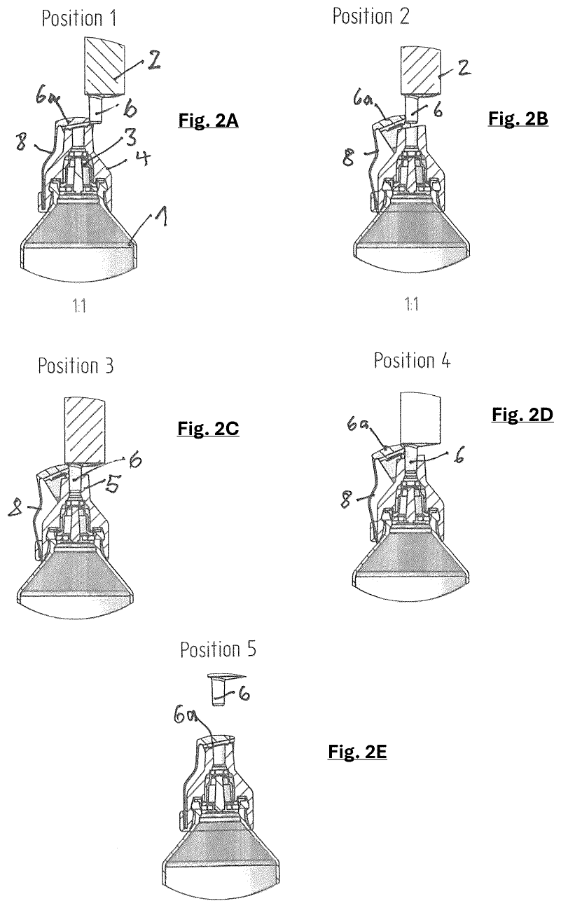

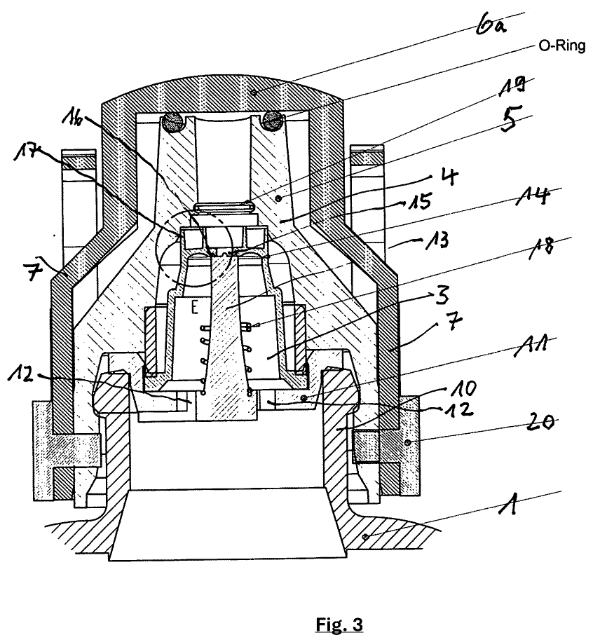

FIG. 2 shows successive steps in the removal of medicinal solution from the multiple dose container 1. In position 1, the disposable syringe 2 is advanced with the male Luer connector 6 to the closure 6a, for which purpose the closure 6a can have an edge indentation, which is not shown in the drawing.

In position 2, the closure 6a is pushed to the side by the Luer connector 6 against the force of the spring 8 so that in position 3 the Luer connector 6 can be sealingly introduced into the Luer connector 5 on the adapter 4. Thereafter the syringe 2 is pulled up in the usual manner, whereby it receives a dose of the medicinal solution from the inner bag of the multiple dose container 1. In position 5, the disposable syringe 2 is removed and the closure 6a is pivoted back into the closed position again by the spring force.

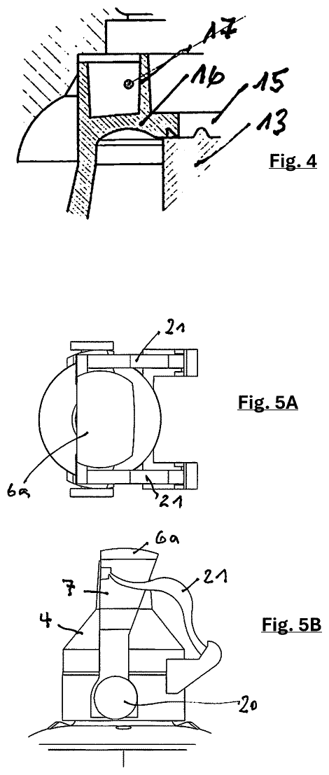

FIG. 3 shows, on an enlarged scale, the adapter 4 with the Luer connector 5. The adapter 4 is snap-connected onto the neck 10 of the multiple dose container 1. The invention is, however, not limited to this and instead of this the adapter could also be screwed or crimpled onto a container neck.

The adapter 4 tapers conically towards the female Luer connector 5, which constitutes the outlet opening of the one-way valve 3 accommodated in the adapter 4. The one-way valve consists of an inner cone 11, which is positioned on the upper edge of the bottle neck 10 and includes openings 12 for the medicinal solution situated in the multiple dose container. The valve inner cone also includes a central valve seat pin 13, which extends in the longitudinal direction of the adapter and resting on whose upper side there is a valve outer cone 14. The valve outer cone 14 includes a central hole 15, the edge region of which sealingly engages the upper side of the valve seat pin 13 in the unactuated state of the valve 3.

The lower edge region of the valve outer cone 14 is fastened sealingly to the valve inner cone 11 and extends upwardly in a conical or bell-shaped manner to its upper wall with the opening 15. Arranged on the upper side of the upper wall 16 is a double row of sealing fins 17, which engage the inner wall of the adapter 4.

In the non-actuated state of the system, i.e. when no medicinal solution is being removed, the valve outer cone 14 forms a sealed closure of the internal space of the multiple dose container 1 since the upper wall 16 of the valve outer cone 14 sealingly engages the upper side of the valve seat pin 13. However, when the disposable syringe 2 is docked with its male Luer connector 6 onto the adapter 4 and the disposable syringe is drawn up, the upper wall 16 of the valve outer cone 14 is lifted away from the upper side of the valve seat pin 13 by the suction force so that medicinal solution is sucked through the openings 12 in the base wall of the valve and through the upper hole 15 in the valve outer cone 14 into the disposable syringe 2.

A silver coil 18 is wound around the valve seat pin 13 as a safety feature against contamination of the medicinal solution and a silver ring 19 is located in the lower region of the female Luer connector 5.

FIG. 3 shows also that the closure 6a of the adapter is integrally connected with its two lateral webs 7, which are pivotally connected to the housing of the adapter 5 by bearing pins 20.

FIGS. 5A and 5B show, in an alternative embodiment, that two curved spring elements 21 connect the webs 7 to the adapter 4.

1. A system for storing a sterile medicinal solution in a multiple dose container and for delivering one dose at a time of the medicinal solution to a disposable syringe, wherein the multiple dose container is an airless container, which consists of a stiff outer container and a contracting inner bag, into which no air enters for pressure balancing when delivering medicinal solution, that fastened to the multiple dose container there is an adapter with a one-way valve and a female Luer container and that the disposable syringe is provided with a matching, male Luer connector, with which it may be docked with the adapter to receive a dose of the medicinal solution by pulling up the disposable syringe.

2. The system as claimed in claim 1, wherein the multiple dose container has a container neck and that the Luer adapter is snap-connected to the container neck.

3. The system as claimed in claim 1, wherein the female Luer connector is a conically broadening outlet passage in the one-way valve.

4. The system as claimed in claim 1, wherein the one-way valve has an elastic valve membrane, which closes the entire inner flow opening of the one-way valve with the exception of a hole, the edge of which valve membrane so rests, in the relaxed state, on a valve seat pin that the one-way valve is sealingly closed.

5. The system as claimed in claim 4, wherein when the docked disposable syringe is pulled up, the valve membrane is lifted up from the valve seat pin and enables the flow of medicinal solution.

6. The system as claimed in claim 1, wherein the outlet opening of the Luer connector on the adapter is closeable by a lid, which is connected to two lateral webs which are pivotally connected to the outer side of the adapter, whereby the lid is furthermore connected to at least one flexible spring arm, which is stressed when the lid is pivoted into the open position.

7. The system as claimed in claim 6, wherein the lid has an edge opening, into which the Luer connector on the disposable syringe can engage, in order to move the lid sideways.

8. The system as claimed in claim 1, wherein a dead volume within the adapter is protected from microbial contamination by an antimicrobial material.
